(19) United States
(12) Patent Application Publication
(10) Pub. No.: US 2016/0169719 A1
(43) Pub. Date: Jun. 16, 2016

(54) Fluid container with fluid intake measurement function

(71) Applicant: Chun Kwong CHOI, FO TAN (HK)

(72) Inventor: Chun Kwong CHOI, FO TAN (HK)

(21) Appl. No.: 14/565,432

(22) Filed: Dec. 10, 2014

(51) Int. Cl. G01F 1/00; G01F 23/26

(52) U.S. Cl. CPC G01F 1/00; G01F 23/263

(57) ABSTRACT

A fluid container with fluid intake measurement function includes a container for receiving and holding fluid, a capacitive sensing means positioned in proximity to an outer surface of the container and configured to sense volume of the fluid in the container, an accelerometer disposed in proximity to the outer surface of the container and configured to sense movement and orientation of the container, and a microprocessor electrically connected with the capacitive sensing means and the accelerometer and configured to calculate a current fluid volume and a fluid intake volume according to output of the capacitive sensing means when output of the accelerometer indicates that the container is placed vertically and indicates no motion.

### BACKGROUND OF THE INVENTION

The present invention relates to a fluid container with fluid intake measurement functionality.

As public awareness for personal health and wellness increases, health care products are gaining in popularity and as such, the demand for devices that can measure and communicate data related to one's personal health are on the rise. While there are many devices that can measure and track activity, sleep and internal metrics like heart-rate, blood-pressure and glucose levels, there are very few products that can measure hydration level or fluid consumption. Those products that can rely on sensor configurations positioned inside a drinking vessel to measure the volume of water consumed. This is problematic and can lead to water or fluid contamination and an overall degradation in accuracy and performance of the sensors themselves.

### BRIEF SUMMARY OF THE INVENTION

In view of the aforementioned disadvantages in the products currently available in the market, the present invention intends to provide a fluid container with fluid intake measurement functionality that achieves greater accuracy and durability without the likelihood of fluid contamination.

To attain this objective, the present invention comprises the following:

a container for receiving and holding fluid;

a capacitive sensing means positioned in proximity to an outer surface of the container and configured to sense volume of the fluid inside the container;

an accelerometer positioned in proximity to the outer surface of the container and configured to sense movement and orientation of the container;

a microprocessor electrically connected with the capacitive sensing means and the accelerometer and configured to calculate a current fluid volume and a fluid intake volume according to the output of the capacitive sensing means when the output of the accelerometer indicates that the container is placed vertically on a flat surface and indicates no motion.

A housing is attached to an outer surface of the container for accommodating the capacitive sensing means, the accelerometer and the microprocessor. As such, the fluid inside the container would not be in direct contact with any of the capacitive sensing means, the accelerometer and the microprocessor, thus preventing contamination of the fluid inside the container and also damage to the capacitive sensing means, the accelerometer and the microprocessor by the fluid inside the container.

A user interface is provided at the housing for receiving input from user and displaying output from the microprocessor.

The user interface may be in form of an LCD for displaying output from the microprocessor for receiving input from the user.

The housing is provided with a battery compartment for receiving batteries which are used to supply power to the microprocessor, the capacitive sensing means, the accelerometer and the user interface.

The capacitive sensing means comprises a plurality of electrically conductive plates arranged in sequence along a vertical direction of the container and attached on a wall of the housing closely adjacent to the outer surface of the container. Alternatively, the capacitive sensing means comprises an elongated electrically conductive plate attached on a wall of the housing closely adjacent to the outer surface of the container and along a vertical direction of the container. Such electrically conductive plates are readily available in the marketplace and thus details of the structure thereof are not repeated herein.

In the present invention, the accelerometer senses movement and orientation of the container, and output corresponding signals to the microprocessor. When the output of the accelerometer indicates that the container is placed vertically on a flat surface and indicates no motion, the microprocessor receives output from the capacitive sensing means to calculate the current fluid volume and fluid intake volume according to the respective capacitance change of each of the electrically conductive plates and output the calculated current fluid volume and fluid intake volume to the user interface. When the output of the accelerometer indicates that the container is tilted or in motion (such as in the case of the user tilting the container to drink the fluid in the container, or the user transporting the container, or in incidences where the container is placed on a tilted surface so that the fluid level is not horizontal), the microprocessor discontinues receiving output from the capacitive sensing means to prevent miscalculation.

### BRIEF DESCRIPTION OF THE DRAWINGS

FIG. 1 illustrates a front view of the present invention according to one embodiment.

FIG. 2 illustrates a cross-sectional view of the present invention according to one embodiment.

### DETAILED DESCRIPTION OF THE INVENTION

The present invention is further described below with reference to but not limited to an embodiment and the accompanying drawings.

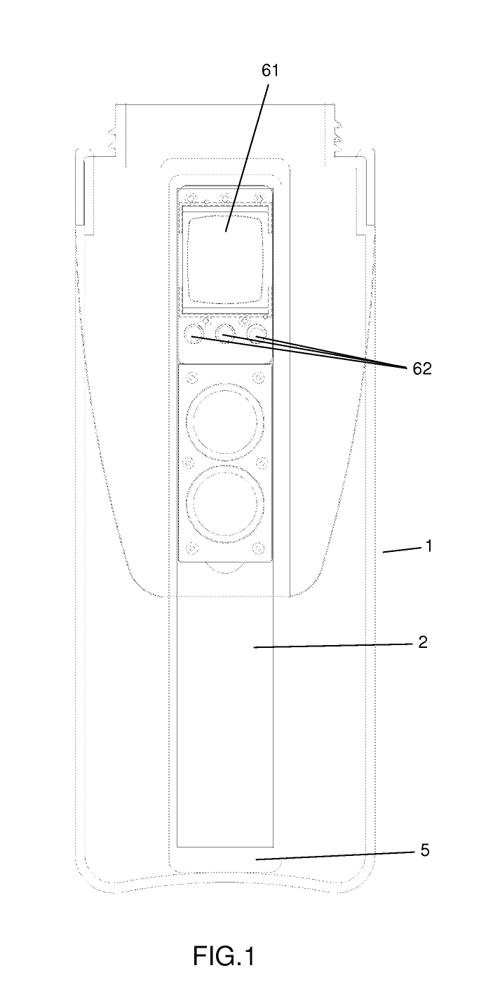

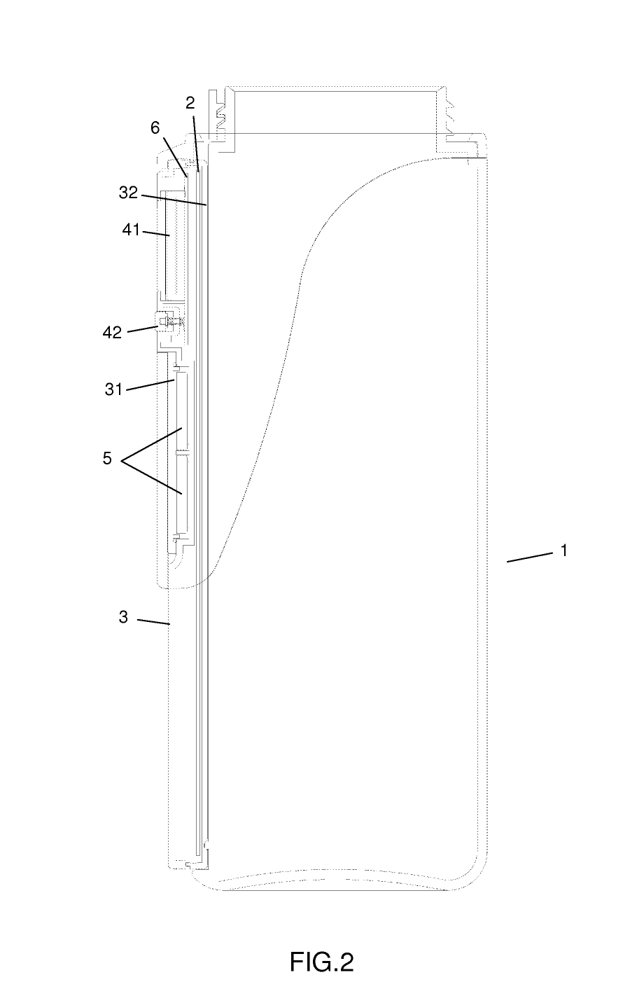

As illustrated in FIGS. 1 and 2, the fluid container of the present embodiment comprises a container 1 for receiving and holding fluid, a capacitive sensing means 2, an accelerometer and a microprocessor. A housing 3 made of PET in this particular embodiment is attached to an outer surface of the container 1 for accommodating the capacitive sensing means 2, the accelerometer and the microprocessor. As such, the fluid inside the container 1 would not be in direct contact with any of the capacitive sensing means 2, the accelerometer and the microprocessor, thus preventing contamination of the fluid inside the container 1 and also damage of the capacitive sensing means 2, the accelerometer and the microprocessor by the fluid inside the container 1.

A user interface in the form of an LCD 41 in this particular embodiment for displaying output from the microprocessor and a plurality of keys 42 for receiving input from user is provided at the housing 3.

The housing 3 is also provided with a battery compartment 31 for receiving batteries 5 which are used to supply power to the microprocessor, the capacitive sensing means 2, the accelerometer and the user interface.

The capacitive sensing means 2 in this embodiment comprises an elongated electrically conductive plate attached on a wall 32 of the housing 3 closely adjacent to the outer surface of the container 1 and along a vertical direction of the container 1, so that the electrically conductive plate is positioned in close proximity to the outer surface of the container 1. The electrically conductive plate is configured to sense volume of fluid in the container 1.

The accelerometer in this embodiment is disposed on a printed circuit board 6 in the housing 3 so that the accelerometer is in proximity to the outer surface of the container 1. The accelerometer is configured to sense movement and orientation of the container 1.

The microprocessor in this embodiment is disposed on the printed circuit board 6. It is electrically connected with the capacitive sensing means 2 and the accelerometer and configured to calculate a current fluid volume and a fluid intake volume according to output of the capacitive sensing means 2 when output of the accelerometer indicates that the container 1 is placed vertically on a flat surface and indicates no motion.

The present embodiment operates as follows:

Initially, the current fluid volume and fluid intake volume are set to 0. Both the current fluid volume and the fluid intake volume are displayed on the LCD 41.

When there is no fluid in the container 1, no electrical charges are sensed by the capacitive sensing means 2 and thus both the current fluid volume and the fluid intake volume remain at 0.

When fluid is poured into the container 1 to cover a part of the capacitive sensing means 2, the capacitive sensing means 2 detects the change in capacitance caused by the presence of the fluid. When the output of the accelerometer indicates that the container 1 is placed vertically on a flat surface and indicates no motion, the microprocessor receives output from the capacitive sensing means 2 to calculate the current fluid volume according to the capacitance change of the capacitive sensing means 2 (e.g. 15 ml in this case). If there is no decrease in the current fluid volume, the fluid intake volume will remain at 0.

When part of the fluid, e.g. 10 ml, is consumed from the container 1, either by the user drinking the fluid directly from the container 1 or pouring the fluid out from the container 1, the output of the accelerometer indicates that the container 1 is tilted or in motion, the microprocessor will stop receiving output from the capacitive sensing means 2 to prevent miscalculation. After the user finishes drinking and places the container 1 on a flat surface, e.g. on a table, the output of the accelerometer indicates that the container 1 is placed vertically and indicates no motion, thus the microprocessor again receives output from the capacitive sensing means 2 to calculate the current fluid volume according to the capacitance change of the capacitive sensing means 2 (e.g. 5 ml in this case). As there is a decrease in the current fluid volume from 15 ml to 5 ml, the microprocessor calculates the volume decreased as the fluid intake volume (10 ml in this case). The microprocessor therefore displays the current fluid volume as 5 ml and the fluid intake volume as 10 ml on the LCD 41.

If the user wishes to reset the fluid intake volume, the user may do so by pressing one of the keys 42 which is predefined as a reset key.

It should be understood that the capacitive sensing means 2, the accelerometer and the user interface are widely available in the marketplace and so no further details on the operation thereof are provided herein.

The above embodiment is a preferred form of the present invention. However, the present invention is not limited by the above embodiment. Any substantive or fundamental changes, modifications, replacements, combinations or simplification within the spirit of the present invention are equivalent alternatives and they are all included in the scope of protection of the present invention.

What is claimed is:

1. A fluid container with fluid intake measurement function which comprises:
a container for receiving and holding fluid;
a capacitive sensing means positioned in proximity to an outer surface of the container and configured to sense volume of the fluid in the container;
an accelerometer positioned in proximity to the outer surface of the container and configured to sense movement and orientation of the container;
a microprocessor electrically connected with the capacitive sensing means and the accelerometer and configured to calculate a current fluid volume and a fluid intake volume according to output of the capacitive sensing means when output of the accelerometer indicates that the container is placed vertically on a flat surface and indicates no motion.

2. The fluid container with fluid intake measurement function as in claim 1, wherein a housing is attached to an outer surface of the container for accommodating the capacitive sensing means, the accelerometer and the microprocessor.

3. The fluid container with fluid intake measurement function as in claim 2, wherein a user interface is provided at the housing for receiving input from user and displaying output from the microprocessor.

4. The fluid container with fluid intake measurement function as in claim 3, wherein the housing is provided with a battery compartment for receiving batteries which are used to supply power to the microprocessor, the capacitive sensing means, the accelerometer and the user interface.

5. The fluid container with fluid intake measurement function as in claim 2, wherein the capacitive sensing means comprises a plurality of electrically conductive plates arranged in sequence along a vertical direction of the container and attached on a wall of the housing closely adjacent to the outer surface of the container.

6. The fluid container with fluid intake measurement function as in claim 2, wherein the capacitive sensing means comprises an elongated electrically conductive plate attached on a wall of the housing closely adjacent to the outer surface of the container and along a vertical direction of the container.
